(19) United States
(12) Patent Application Publication
(10) Pub. No.: US 2008/0200895 A1
(43) Pub. Date: Aug. 21, 2008

(54) Tampon having three-dimensional apertured overwrap

(76) Inventors: Ryo Minoguchi, Cincinnati, OH (US); Kelyn Anne Arora, Cincinnati, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(21) Appl. No.: 11/708,296

(22) Filed: Feb. 20, 2007

(51) Int. Cl. A61F 13/20

(52) U.S. Cl. 604/385.17

(57) ABSTRACT

Tampons having a three-dimensional apertured overwrap. The tampons can include a compressed absorbent member having an inner region and an exterior surface. The compressed absorbant member has one or more absorbent materials. A fluid pervious overwrap covers at least a portion of the exterior surface of the compressed absorbent member. The fluid pervious material of the overwrap is a nonwoven material having three-dimensional apertures therein. A process for making a tampon is also provided.

### FIELD OF THE INVENTION

The invention relates to tampons having a three-dimensional apertured overwrap and more particularly to tampons having a three-dimensional nonwoven apertured overwrap.

### BACKGROUND OF THE INVENTION

A wide variety of absorbent catamenial tampons have long been known. While it has been found that these tampons perform their intended function reasonably well, such as, e.g., leakage protection, the post-use appearance of these tampons can have a negative impact on the user. Until recently it was not appreciated that the visual impact on the user of the post-use tampon was an important design criteria for tampons. In fact, it has been discovered that the post-use appearance is an important factor to consider in affecting the user's total use experience.

As such, it would be desirable to provide a tampon having improved leakage protection and an improved visual appearance after use.

### SUMMARY OF THE INVENTION

Tampons having a three-dimensional apertured overwrap are provided. The tampons can include a compressed absorbent member having an exterior surface and including one or more absorbent materials. The tampons can also include an overwrap covering at least a portion of the exterior surface of the compressed absorbent member. In certain embodiments, the overwrap can include a fluid pervious material that can be a nonwoven material having three-dimensional apertures therein.

The tampon can include a compressed absorbent member having an exterior surface and including one or more absorbent materials and an overwrap covering at least a portion of the exterior surface of the compressed absorbent member. The overwrap can include a fluid pervious material that can be a nonwoven material having a material thickness. In certain embodiments, the nonwoven material can have three-dimensional apertures therein, the three-dimensional apertures each having a macroscopic opening with one or more sidewalls proximate the macroscopic opening, the one or more sidewalls projecting generally outwardly from the surface of the nonwoven material under about zero compression and substantially extending around the periphery of the macroscopic opening, the one or more sidewalls having a sidewall height. The ratio of sidewall height to material thickness can be greater than 1. A process for making a tampon is also provided.

### BRIEF DESCRIPTION OF THE DRAWINGS

FIG. 1 is a perspective view of one embodiment of the present invention.

FIG. 2 is a plan view of one embodiment of the present invention.

FIG. 3 is a cross-sectional view of a three-dimensional apertured nonwoven overwrap material.

FIG. 4 is a cross-sectional view of a two-dimensional apertured nonwoven overwrap material.

FIG. 5 is a schematic representation of a rotary knife apparatus (RKA) and process.

FIG. 6 is a magnified cross-sectional view of one embodiment of the present invention.

### DETAILED DESCRIPTION OF THE INVENTION

The present invention relates to tampons that include a three-dimensional apertured nonwoven overwrap. Apertures in the overwrap can contribute to better fluid acquisition of fluid, including viscous fluid components of menses, such as, e.g., by providing unimpeded fluid pathways to and into the absorbent member. By providing for preferential fluid paths into the absorbent member, the overwrap can remain relatively free of fluid, particularly viscous fluid, or fluid having solids components, such as menses. Apertures can also permit the fluid, such as menses, to penetrate deeper into the article to improve the masking property of the article. This is believed to be due to the absorbed fluid being offset a certain distance from the surface of the overwrap. Therefore, a fluid pervious apertured overwrap can contribute to an overall cleaner post-use appearance.

Surprisingly, apertures with three-dimensionality, such as, e.g., an overwrap having apertures having a greater thickness proximate an aperture than at a non-apertured point on the overwrap under zero compression, can provide improved leakage protection and an improved post-use appearance in a finished tampon, such as, e.g., compared to non-apertured overwraps or overwraps having two-dimensional apertures. For example, apertures having three-dimensionality can tend to remain open instead of closing during the tampon forming process and use, which can improve fluid permeation through the overwrap into the absorbent material of the tampon, as well as provide improved contrast between the overwrap and the stained absorbent material post-use. In certain embodiments, an overwrap having three-dimensional apertures can increase retention of fluid within the tampon during removal from a user's body.

In certain embodiments, the overwrap can comprise an apertured fluid pervious material that can be hydrophobic or rendered hydrophobic relative to the compressed absorbent member, such that absorbed fluid is attracted to and remains in the absorbent member. Because of the relatively poor wicking propensity of the hydrophobic overwrap, the overwrap can tend to remain free of fluid as the fluid is preferentially partitioned into the absorbent member, giving a cleaner visual appearance to the post-use article.

As used herein the term “tampon” refers to any type of absorbent structure that is inserted into the vaginal canal for the absorption of fluid therefrom. Typically, tampons are constructed from an absorbent material that has been compressed into a vaginally insertable shape.

As used herein the terms “pledget” or “tampon pledget” are intended to be interchangeable and refer to a construction of absorbent material prior to the compression of such construction into a tampon.

As used herein the terms “vaginal cavity,” “within the vagina,” and “vaginal interior,” are intended to be synonymous and refer to the internal genitalia of the human female in the pudendal region of the body. The term “vaginal canal” as used herein is intended to refer to the space located between the introitus of the vagina (sometimes referred to as the sphincter of the vagina) and the cervix and is not intended to include the interlabial space, including the floor of vestibule. The externally visible genitalia generally is not included within the term “vaginal canal” as used herein.

As used herein “fluid pervious” refers to the property of a material and can be characterized by the ability to carry fluid or moisture, such as by capillary action, prior to any post-processing step, such as aperturing. Therefore, for example, an untreated woven or nonwoven material is fluid pervious and a thermoplastic film is not. A nonwoven material can permit fluid flow via the interstices between fibers, such as, e.g., by capillary action and/or via a pressure differential from one side of the nonwoven to the other such as the pressure experienced by a tampon in use.

As used herein “aperture” refers to a macroscopic opening or “hole” as distinct from inherent pores or interstices of fluid pervious materials, such as inherent pores or interstices of foams or nonwoven materials, for example. A macroscopic opening is visible to the naked eye of an observer having 20/20 vision at a distance of 45 cm.

As used herein “three-dimensional aperture” refers to an aperture having a greater thickness proximate an aperture than at a non-apertured point on the overwrap under zero compression. For example, in certain embodiments, a three-dimensional aperture can include a macroscopic opening or hole having one or more protuberances or sidewalls projecting generally outwardly from the surface of the web under zero compression. In certain embodiments, a three-dimensional aperture can include one or more sidewalls substantially extending outwardly form the surface of the web around the periphery of the aperture under zero compression. A three-dimensional aperture typically has a ratio of sidewall height to material thickness greater than one, such as, e.g., greater than about 1.5, greater than about 2, greater than about 2.5, or greater than about 3.

As used herein, “vaginally insertable shape” refers to the geometrical form of the absorbent tampon after compression. The tampon can be compressed into a generally cylindrical configuration in the radial direction along the longitudinal and/or lateral axes, axially, or in both the radial and axial directions. An example of a typical compressed tampon is one which is about 10-16 mm wide and about 30-55 mm long depending on absorbency. While the tampon may be compressed into a substantially cylindrical configuration, other shapes are possible. These may include shapes having a cross section that can be described as rectangular, triangular, trapezoidal, semi-circular, hourglass, or other suitable shapes.

As used herein, a first material can “substantially cover” a second material when the first material covers at least about 75%, such as, e.g., at least about 90%, of the surface area of the second material.

The term “joined” or “attached” as used herein, encompasses configurations in which a first element is directly secured to a second element by affixing the first element directly to the second element, configurations in which the first element is indirectly secured to the second element by affixing the first element to intermediate member(s) which in turn are affixed to the second element, and configurations in which first element is integral with second element, i.e., first element is essentially part of the second element.

The term “rolled” as used herein, refers to the configuration of the compressed absorbent member after winding the absorbent material in a spiral round and round upon itself.

The term “folded” as used herein, refers to the configuration of the compressed absorbent member that may be incidental to lateral compaction of the absorbent material or may purposely occur prior to a compression step. Such a configuration is readily recognizable, for example, when the absorbent material abruptly changes direction such that one part of the absorbent material bends and lies over another part of the absorbent material.

Tampons having a fluid pervious overwrap covering at least a portion of the exterior surface of the compressed absorbent member are provided. In certain embodiments, the overwrap can include a fluid pervious material that can be a nonwoven material having apertures, such as, e.g., three-dimensional apertures, therein. In certain embodiments, the overwrap can substantially permeate the folds and interstices, e.g., an inner region, of the compressed absorbent member and/or can extend beyond the withdrawal end to form a skirt. The nonwoven material can be hydrophilic or hydrophobic; however, in certain embodiments, the nonwoven material can be hydrophobic or rendered hydrophobic.

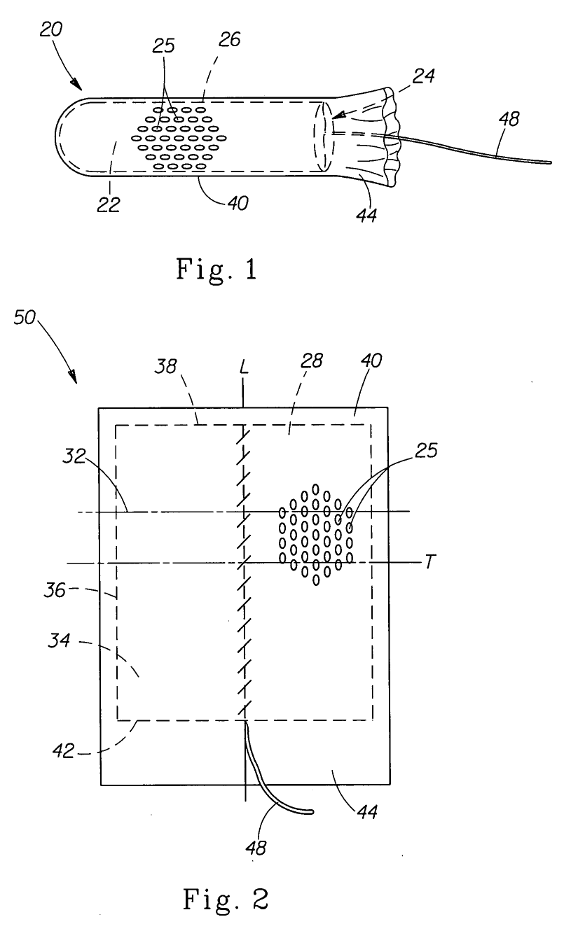

FIG. 1 shows one embodiment of an absorbent tampon 20 of the present invention. A compressed absorbent member 22 (sometimes referred to as the “absorbent core”) of the tampon 20 has an exterior surface 26. The compressed absorbent member 22 has an inner region 24. The exterior surface 26 of the compressed absorbent member 22 is substantially covered by a fluid pervious overwrap 40. The fluid pervious overwrap 40 can comprise a plurality of apertures 25 that can permit improved fluid flow of viscous fluid to and into the compressed absorbent member 22. The fluid pervious overwrap can also extend to provide a skirt portion 44. In one embodiment, the tampon 20 can include a withdrawal means 48, such as a string.

FIG. 2 shows one embodiment of a pledget 50 in a flat-out configuration, prior to being folded or rolled and compressing into a tampon 20, such as the tampon shown in FIG. 1. Fluid pervious overwrap 40, shown in FIG. 2 as having apertures 25, substantially covers the absorbent material 28. The absorbent material 28 has a first surface 34 opposed to a second surface 36 and an insertion end 38 opposed to a withdrawal end 42. The absorbent material 28 has both a longitudinal axis and a transverse axis indicated by the lines marked “L” and “T” respectively. Because the fluid pervious overwrap 40 can be wrapped in various configurations, the width (or length) of fluid pervious overwrap 40 may vary. The dimensions of the fluid pervious overwrap 40 may be greater than, less than, or equal to the dimensions of the longitudinal or transverse axes of the absorbent material being wrapped.

At least a portion of, such as, e.g., all of, the first surface 34 and/or opposed second surface 36 of the absorbent material 28 can be covered by fluid pervious overwrap 40 prior to compressing into compressed absorbent member 22. The fluid pervious overwrap can be positioned by folding around the insertion end 38 of the absorbent material 28. The fluid pervious overwrap can overlap at the region 32, for example. The overlapping portions in region 32 need not be sealed, partially sealed, or otherwise joined, although such joining can be done.

The fluid pervious overwrap can comprise a fibrous nonwoven material comprising natural, synthetic, or a blend of natural and synthetic fibers. Suitable synthetic fibers can include, e.g., fibers such as polyester, polyolefin, nylon, polypropylene, polyethylene, polyacrylic, cellulose acetate, polyhydroxyalkanoates, aliphatic ester polycondensates, bicomponent fibers and/or mixtures thereof. Natural fibers can include, e.g., rayon and those commonly known to be non-synthetic and of natural origin such as cotton. The fibers can have any suitable cross-sectional shape, such as, e.g., round, tri-lobal, multi-lobal, delta, hollow, ribbon-shaped, and/or any other suitable shape, or mixtures thereof. Fibers with any suitable diameter can be used, such as, e.g., from about 0.5 to about 50 microns, such as, e.g., from about 1 to about 30 microns, such as, e.g., from about 10 to about 25 microns. Fiber diameter can be determined using any suitable means; however, for non-round fibers, diameter can typically be determined by reference to the diameter of a fiber with the same cross-sectional area as the non-round fiber.

The fluid pervious overwrap can be made by any number of known techniques. Suitable techniques include, for example, carding, meltblowing, spunbonding, spunlacing, air laying, and the like. In certain embodiments, the fluid pervious overwrap can be formed using bonding methods, such as, e.g., thermal, ultrasonic, resin, through-air bonding, hydroentangling, and/or needling.

The basis weight of the nonwoven overwrap prior to forming apertures can be any suitable weight, such as, e.g., from about 10 to about 60 grams per square meter (gsm), such as, e.g., from about 15 to about 30 gsm. Synthetic fibers, if used, can have hydrophobic and/or hydrophilic finishes, although, as mentioned above, in certain embodiments, the fibers of the nonwoven can be rendered hydrophobic relative to the absorbent member.

The overwrap can be biodegradable, or bio-disentegratable. Suitable biodegradable polymers can include, e.g., biodegradable polymers such as PHAs, PLAs, starch compositions and other biodegradable polymers described in U.S. Publication 2002/0188041-A1. In certain embodiments, the fluid pervious overwrap can comprise rayon, a rayon/cotton blend, or a blend of rayon and cotton with polymeric fibers. The rayon or rayon/cotton blend can be then treated to be hydrophobic.

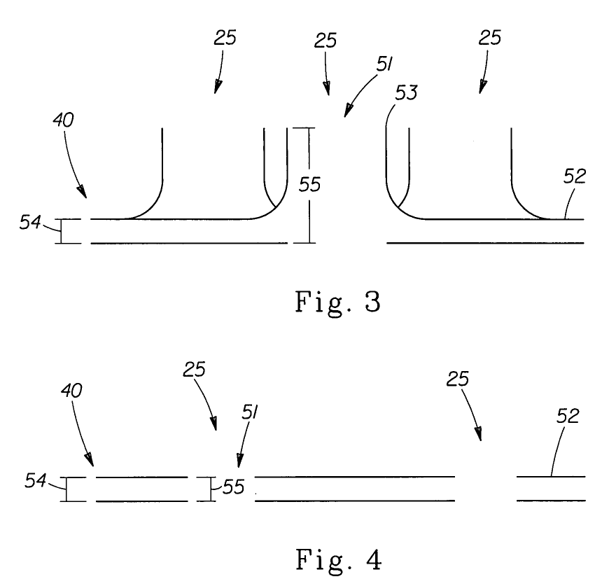

The fluid pervious overwrap can be a three-dimensional apertured nonwoven material. As shown in FIG. 3, in certain embodiments, the fluid pervious overwrap 40 can have apertures 25 that can be three-dimensional. The apertures 25 can include a macroscopic opening 51 having one or more protuberances or sidewalls 53 projecting generally outwardly from the overwrap surface 52, such as, e.g., one or more sidewalls substantially extending around the periphery of the aperture, under zero compression. In certain embodiments, the three-dimensional aperture can be in the form of a cone, such as, e.g., in the general shape of a volcano. The fluid pervious overwrap 40 can have a material thickness 54 and a sidewall height 55. A three-dimensional apertured nonwoven overwrap can typically have a ratio of sidewall height 55 to material thickness 54 greater than one, such as, e.g., greater than about 1.5, greater than about 2, greater than about 2.5, or greater than about 3. In certain embodiments, substantially all of the apertures on the nonwoven material can be three-dimensional.

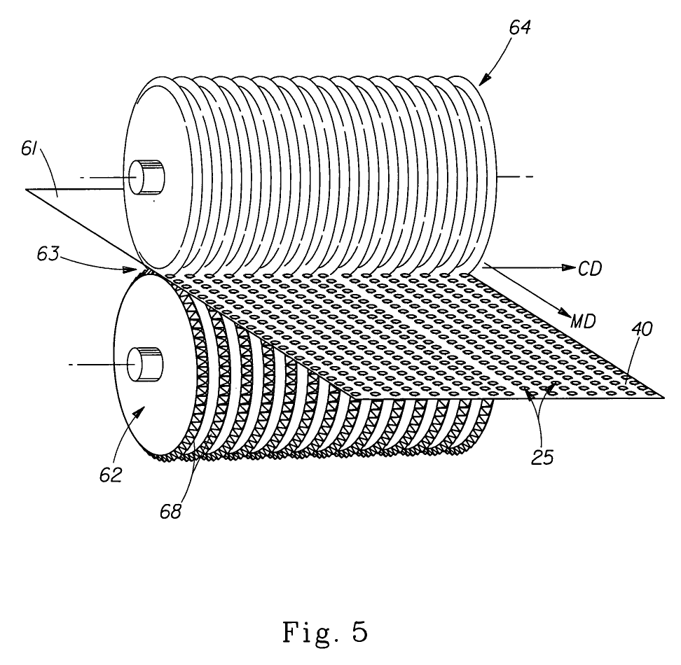

Two-dimensional apertured nonwoven overwraps, on the other hand, can typically have a ratio of sidewall height 55 to material thickness 54 of about 1. As shown in FIG. 4, a two-dimensional apertured nonwoven material can have apertures 25 that include a macroscopic opening 51. The fluid pervious overwrap 40 can have a material thickness 54 and a sidewall height 55. In certain embodiments, a two-dimensional apertured nonwoven overwrap can have a material thickness 54 and a sidewall height 55 that are approximately the same, that is, the two-dimensional apertures can have substantially no protuberance projecting generally outwardly from the overwrap surface 52. Typical methods for forming two-dimensional apertured nonwoven overwraps, include, e.g., hydroentangling on a suitable forming screen, stretch aperturing, or other methods.

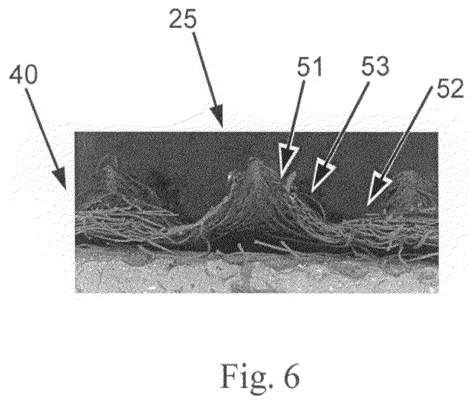

Three-dimensional apertures can be formed in any suitable manner. In certain embodiments, three-dimensional apertures can be made by puncturing a nonwoven overwrap, such as, e.g., using “rotary knife aperturing” (RKA), such as, e.g., described in U.S. Appln. No. 2006/0087053. RKA utilizes a pair of intermeshing rolls that are typically steel. The rolls have interengaging grooves and teeth that are generally pointed at the distal end, as shown in FIG. 5. Teeth 68 can be sharpened to cut through an overwrap material 61 to produce a three-dimensionally apertured fluid pervious overwrap 40. As shown in FIG. 5, an unmodified overwrap material 61 can be fed from a supply roll into the nip 63 of opposed forming rolls 62 and 64. As overwrap 61 passes through nip 63, the teeth 68 of roll 62 can form three-dimensional apertures 25 in overwrap 61, resulting in three-dimensionally apertured fluid pervious overwrap 40. RKA can be employed using any suitable equipment and parameters for forming three-dimensional apertures, such as, e.g., parameters disclosed in co-pending, commonly owned patent application US 2006/0087053, filed Oct. 13, 2005. A magnification of a three-dimensional apertured nonwoven overwrap 40 having one or more apertures 25 having a macroscopic opening 51, one or more sidewalls 53 projecting generally outwardly from the overwrap surface 52 is shown in FIG. 6. Alternatively, or in addition, three-dimensional apertures can be made by laying or impressing fibers over a three-dimensionally structured screen or mesh such as, e.g., described in U.S. Pat. Nos. 4,024,612; 5,503,715; and/or 6,270,623, and/or by hot pin punching.

Apertures can be of virtually any shape and size, as long as the overwrap can provide the function of covering the absorbent member after in-use expansion, and provide for a masking effect after use. In certain embodiments, apertures can be generally round or oblong shaped, in a regular pattern of spaced apart openings. The apertures can each have any suitable area, such as, e.g., an area of from about 0.3 mm² to about 2 mm², such as, e.g., about 1 mm², and can form an open area of between about 1% and about 25%, such as, e.g., between about 2% and about 20%, such as, e.g., between about 10% and about 15%. In certain embodiments, the apertures can be provided in non-repeating and/or non-regular patterns that can be random and/or can have various shapes and sizes.

In certain embodiments, the apertures can be a three-dimensional aperture having a macroscopic opening having one or more sidewalls projecting generally outwardly from the surface of the web under zero compression. The one or more sidewalls can have any suitable height. In certain embodiments, the one or more sidewalls can have a height suitable to provide a ratio of sidewall height to thickness of the overwrap material of greater than one, such as, e.g., greater than about 1.5, greater than about 2, greater than about 2.5, or greater than about 3.

As set forth herein, in certain embodiments, the fluid pervious overwrap can be hydrophobic relative to the compressed absorbent member. Hydrophobicity can be inherent due to the material properties of the fluid pervious overwrap material, or the fluid pervious overwrap can be rendered hydrophobic by suitable treatment of an otherwise hydrophilic material. For example, the fluid pervious overwrap can comprise one or more fibers that are inherently more hydrophobic than the compressed absorbent member, such as, e.g., polypropylene spunbond and/or conjugate fibers, such as bicomponent polyethylene/polypropylene fibers and/or bicomponent polyethylene/polyester fibers.

Alternatively, or in addition, the overwrap can contain hydrophilic fibers, such as, e.g., rayon or a blend of rayon/cotton, that can, in certain embodiments, be rendered hydrophobic by a suitable treatment. Any suitable treatment can be employed, such as, e.g., a coating of a suitable material sufficient to render the fluid pervious overwrap sufficiently hydrophobic. For example, surface treatments can include applied coatings of silicone, such as Dow Corning 108® silicone, available from the Dow Corning Co. Inc., Midland, Mich.; or Sucrose Esters of Fatty Acids (SEFA), available from the Procter & Gamble Co., Inc., Cincinnati, Ohio, polyolefin waxes, or NALAN® available from DuPont, Wilmington Del. Such coatings can render the fluid pervious overwrap hydrophobic, or highly hydrophobic. The application of a suitable surface treatment can be achieved by spraying, slot coating, immersion and other suitable methods. The amount of coating can be varied as needed to render the fluid pervious overwrap sufficiently hydrophobic relative to the compressed absorbent member. In one embodiment, a 1% by weight coating of SEFA can be utilized. Sufficient relative hydrophobicity is achieved when fluid such as menses is absorbed into the compressed absorbent member in use, and is sufficiently desorbed from the fluid pervious overwrap so as to partition the fluid into the core and away from the overwrap.

The fluid pervious overwrap can be joined to the absorbent material by any variety of means. The fluid pervious overwrap can be joined to itself or to the absorbent material. For example, one portion of fluid pervious overwrap can be joined to an opposed portion of the fluid pervious overwrap or the absorbent member using any suitable adhesive or heat/pressure bonding means. Such adhesive can extend continuously along the length of attachment or it can be applied in a non-continuous fashion at discrete intervals. Heat bonding includes thermally bonding, fusion bonding, or any other suitable means for joining such materials. Alternatively, the fluid pervious overwrap may be joined to the absorbent material along with the withdrawal cord by stitching as shown in FIG. 2. Such stitching may use natural or synthetic thread.

The compressed absorbent member can be formed in any suitable manner. In certain embodiments, the absorbent material can be joined to the fluid pervious overwrap and can be rolled and/or folded, compressed and optionally heat conditioned in any suitable conventional manner. In certain embodiments, after rolling or folding and compression, the apertured fluid pervious overwrap can cover the exterior surface of compressed absorbent member and can also be embedded in the interior folds of the compressed absorbent member. That is, in certain embodiments, the apertured fluid pervious overwrap can permeate the interior of the compressed absorbent member.

The absorbent material can be any suitable shape, size, material, or construction prior to compression and/or shaping. For example, the pledget can include a rolled, tubed, or flat construction of an absorbent that can be a circle, an oval, a semi-circle, a triangle, a chevron shape, an H shape, a bow-tie shape, or any other suitable shape, such as, e.g., shapes described in, for example, U.S. Pat. Nos. 3,738,364; 5,911,712; 6,740,070; 6,887,266; and 6,953,456. A typical size for absorbent material prior to compression can be from about 30 mm to about 100 mm in length and from about 30 mm to about 80 mm in width. The typical range for the overall basis weight of the absorbent material 28 is from about 150 gsm to about 1250 gsm depending upon desired absorbent capacity. The materials for the tampon can be formed into a fabric, web, or batt that is suitable for use in the absorbent material by any suitable process such as airlaying, carding, wetlaying, hydroentangling, needling or other known techniques.

The absorbent material can be a laminar structure comprised of integral or discrete layers. In other embodiments, the pad need not have a layered structure at all. The absorbent material may comprise a folded structure or may be rolled. The resulting compressed absorbent member of the tampon can be constructed from a wide variety of liquid-absorbing materials commonly used in absorbent articles. Such materials include, for example, rayon (such as GALAXY rayon (a tri-lobed rayon) or DANUFIL rayon (a round rayon), both available from Kelheim Fibres GmbH of Kelheim, Germany), cotton, folded tissues, woven materials, nonwoven webs, synthetic and/or natural fibers or sheeting, comminuted wood pulp, which is generally referred to as airfelt, foams, or combinations of these materials. Examples of other suitable materials include: creped cellulose wadding; meltblown polymers including coform; chemically stiffened, modified or cross-linked cellulosic fibers; synthetic fibers such as crimped polyester fibers; peat moss; foam; tissue including tissue wraps and tissue laminates; or any equivalent material or combinations of materials, or mixtures of these. Additionally, superabsorbent materials, such as superabsorbent polymers or absorbent gelling materials can be incorporated into the tampon.

Any suitable pressures and temperatures for compression can be used. In certain embodiments, the absorbent material and the fluid pervious overwrap can be compressed in the radial direction and optionally axially by any suitable means. While a variety of techniques are known and acceptable for these purposes, a tampon compressor machine available from Hauni Machines, Richmond, Va., can be suitable.

In certain embodiments, all or a portion of the tampon can be compressed into a substantially cylindrical configuration, however, other shapes are possible. These can include shapes having a cross section or cross-section element that can be described as rectangular, triangular, trapezoidal, semi-circular, hourglass, or other suitable shapes.

In certain embodiments, the tampon can be a tampon having a radially compressed rolled construction. The tampon can be constructed by rolling and radially compressing a pledget. The pledget can be rolled around a mandrel then compressed with or without the mandrel. In certain embodiments, a cavity left behind after the mandrel is removed can provide a finger pocket. The tampon can also be constructed by pressing a pledget, such as, for example, a cylindrical pledget, in forming dies with a pushrod. A cavity that can be a finger pocket can be formed in part of the blank pressed against a convex in the forming dies or the pushrod.

The tampon can additionally comprise a withdrawal means. The withdrawal means could be joined to the tampon and graspable by the user for removal after use. The withdrawal means can be joined to at least the compressed absorbent member and extends beyond the withdrawal end. Any suitable withdrawal means, such as, e.g., string, can be used as a suitable withdrawal mechanism. In addition, the withdrawal means can take on other forms such as a ribbon, loop, tab, or the like. The withdrawal means may be integral with the absorbent material. Alternatively, or in addition, the withdrawal means can be attached in any suitable manner including sewing, adhesive attachment, or a combination of known bonding methods. The withdrawal means can be joined to any suitable location on the tampon. The tampon can also or alternatively include one or more secondary absorbent members, such as, for example, a mass of secondary absorbent material attached to the withdrawal cord proximate the withdrawal end of the tampon. Suitable secondary absorbent members are described in, e.g., U.S. Pat. No. 6,258,075.

While several methods of making the tampon of the present invention should be apparent to one of skill in the art in light of the disclosure herein, following is a description of one method of making a tampon of the present invention.

In certain embodiments, the process for making a tampon can include the steps of providing an absorbent material having a first surface opposed to a second surface and an insertion end opposed to a withdrawal end, and providing a fluid pervious overwrap that can be a nonwoven material having a material thickness, the nonwoven material having three-dimensional apertures therein. The fluid pervious overwrap can be wrapped or folded about absorbent material to cover at least a portion of, and/or or substantially cover, one or more surfaces. The apertured fluid pervious overwrap can extend beyond the withdrawal end of the absorbent material to form a skirt portion and/or a withdrawal means such as, e.g., a string can be attached, such as by stitching, to the absorbent material. The wrapped absorbent can be rolled or folded and/or compressed to form a compressed absorbent member having a vaginally insertable shape. Upon compression, the fluid pervious overwrap can cover at least a portion and/or substantially cover the exterior surface of the compressed absorbent member.

The nonwoven material can be apertured at any suitable point during the process for making a tampon. For example, in certain embodiments, the nonwoven material can be apertured prior to the tampon forming process, such as, e.g., using separate machinery. Alternatively, the nonwoven material can be provided on the tampon converting line in unmodified form and can be apertured during the tampon forming process, such as, e.g., prior to, during, or after the formation of the tampon pledget, but prior to joining the overwrap to the tampon pledget and compression of the pledget into a compressed absorbent member. In certain embodiments, the nonwoven material can be three-dimensionally apertured on the tampon converting line and the apertures can be registered to the tampon pledget prior to compression.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A catamenial tampon comprising:
a compressed absorbent member having an inner region and an exterior surface, the compressed absorbent member comprising one or more absorbent materials;
an overwrap covering at least a portion of the exterior surface of the compressed absorbent member, the overwrap comprising a fluid pervious material;
wherein the fluid pervious material of the overwrap is a nonwoven material having a material thickness and three-dimensional apertures therein.

2. The tampon of claim 1, wherein the three-dimensional apertures comprise a macroscopic opening having one or more sidewalls proximate the macroscopic opening, the one or more sidewalls projecting generally outwardly from the surface of the nonwoven material under about zero compression.

3. The tampon of claim 2, wherein the one or more sidewalls have a sidewall height, and wherein the ratio of sidewall height to material thickness is greater than 1.

4. The tampon of claim 3, wherein the ratio of sidewall height to material thickness is greater than about 1.5.

5. The tampon of claim 3, wherein the ratio of sidewall height to material thickness is greater than about 2.

6. The tampon of claim 1, wherein the overwrap is hydrophobic or rendered hydrophobic relative to the compressed absorbent member.

7. The tampon of claim 1, wherein the apertures have a mean area of about 0.3 mm² to about 2 mm².

8. The tampon of claim 1, wherein the apertures are provided in a predetermined portion of the overwrap, the predetermined portion being less than the entire overwrap.

9. A catamenial tampon comprising:
a compressed absorbent member having an inner region and an exterior surface, the compressed absorbent member comprising one or more absorbent materials; and
an overwrap covering at least a portion of the exterior surface of the compressed absorbent member, the overwrap comprising a fluid pervious material;
wherein the fluid pervious material is a nonwoven material having a material thickness and three-dimensional apertures therein, the three-dimensional apertures comprising a macroscopic opening having one or more sidewalls proximate the macroscopic opening,
wherein the one or more sidewalls project generally outwardly from the surface of the nonwoven material under about zero compression, substantially extend around the periphery of the macroscopic opening, and have a sidewall height, and
wherein the ratio of sidewall height to material thickness is greater than 1.

10. The tampon of claim 9, wherein the ratio of sidewall height to material thickness is greater than about 2.

11. The tampon of claim 9, wherein the ratio of sidewall height to material thickness is greater than about 3.

12. The tampon of claim 9, wherein the tampon is a digital tampon.

13. The tampon of claim 9, where the apertures are formed by puncturing the overwrap.

14. The tampon of claim 13, wherein the apertures are formed using rotary knife aperturing.

15. The tampon of claim 13, wherein the overwrap is hydrophobic or rendered hydrophobic relative to the compressed absorbent member.

16. A process for making a tampon comprising the steps of:
(a) providing an absorbent material having a first surface opposed to a second surface and an insertion end opposed to a withdrawal end;
(b) providing a fluid pervious overwrap that is a nonwoven material having a material thickness, the nonwoven material having three-dimensional apertures therein;
(c) creating a wrapped absorbent by covering at least a portion of the first surface and/or second surface of the absorbent material with the fluid pervious overwrap; and
compressing the wrapped absorbent to form a compressed absorbent member having a vaginally insertable shape, the compressed absorbent member having an inner region and an exterior surface.

17. The process of claim 16, further comprising the step of aperturing the fluid pervious overwrap using rotary knife aperturing prior to step (b) or (c).

18. The process of claim 17, wherein the process is performed on a converting line and the nonwoven material is three-dimensionally apertured on the converting line.

19. The process of claim 17, wherein the ratio of sidewall height to material thickness is greater than about 2.

20. The process of claim 17, wherein the ratio of sidewall height to material thickness is greater than about 3.
